(19) United States
(12) Patent Application Publication
(10) Pub. No.: US 2007/0106233 A1
(43) Pub. Date: May 10, 2007

(54) Systems and methods for dilating and accessing body lumens

(75) Inventors: Alex Huang, Menlo Park, CA (US); Robert S. Behl, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PERCUTANEOUS SYSTEMS, INC., Mountain View, CA (US)

(21) Appl. No.: 11/256,562

(22) Filed: Oct. 20, 2005

(51) Int. Cl. A61M 5/00

(52) U.S. Cl. 604/271; 606/108; 604/104

(57) ABSTRACT

A luminal access and dilation system comprises an access sheath, an obturator, and an evertable lubricous sleeve. The evertable lubricous sleeve is initially stowed within an axial passage of the obturator and is everted from the axial passage over the exterior of the obturator and the axis sleeve as the system is advanced into a body lumen. The obturator may be removed from the access sheath to provide a central passage for access to the body lumen or target body cavity.

### BACKGROUND OF THE INVENTION

1. Field of the Invention

The present invention relates generally to medical apparatus and methods for using such apparatus. In particular, the present invention relates to methods and systems for atraumatically dilating and accessing body lumens.

A wide variety of catheters, probes, and tubular structures are used in an almost unlimited number of medical procedures and protocols. Of particular interest herein, medical dilators and access tubes are used to treat and access body lumens, for example, a patient's urethral canal which leads to the bladder. Simple catheters, referred to as urinary catheters, may be inserted into the bladder for drainage or to provide access into the bladder for therapy and other purposes. In some instances, it is desirable to advance an access sheath through the urethra into the bladder to permit the introduction of viewing scopes and other instruments for accessing the ureter or for performing procedures in the bladder. In other cases, dilators are introduced into the urethra itself in order to treat strictures and other inclusions which may be present.

Passage of instruments through the urethra for any of these purposes presents a number of risks to the patient, including the introduction of bacteria which can cause infections, irritation of the urethral wall which can cause discomfort and, in the worst cases, cause mechanical injury to the walls, and the like. In other instances, strictures or occlusions within the urethra can make introduction of an access tube or dilator difficult or in some cases impossible.

To overcome at least some of these problems, Memcath LLC. of St. Paul, Minn., has developed the Memcath™ Intermittent Urology Catheter which uses a modified PTFE film sheath which everts from the interior of the catheter over the exterior as the catheter is introduced. As illustrated in FIG. 1, the Memcath™ catheter 10 has a tubular PTFE film membrane 12 which is initially stowed within the lumen 14 of the catheter. The membrane extends out a distal end of the catheter 10 and has an everting section 18 which attaches to a ring 20 which can slide over the exterior surface of the catheter. In this way, as the catheter 10 is advanced into a body lumen, such as the urethra, the tubular membrane 10 will be pulled around the distal end 16 of the catheter to cover the exterior of the catheter as it advances. Since the ring 20 is held stationary relative to the body lumen, the membrane, once it is deployed, will also remain stationary, reducing the risk of trauma to the luminal wall and preventing the propagation of bacteria and other pathogens upward into the body lumen.

While the Memcath™ design is fundamentally sound, it does suffer from certain shortcomings. In particular, because of its blunt distal tip, the Memcath™ catheter is not optimal for use in dilation of the urethra or other body lumens. Additionally, the blunt and open leading end can make it difficult for the Memcath™ catheter to pass strictures or other occlusions or obstructions within the urethra or other body lumen. As a still further shortcoming, the Memcath™ catheter is not designed for use with guidewires which are helpful in both passing obstructions and accessing otherwise difficult urethras and other body lumens.

For these reasons, it would be desirable to provide improved apparatus and methods for accessing and optionally dilating the urethra and other body lumens. It would be particularly desirable if the apparatus and methods provided for atraumatic dilation of the urethra and other body lumens. It would be further desirable if the apparatus could be introduced through a urethra or other body lumen even in the presence of strictures and other occlusions and obstructions. It would be still further desirable to provide for introduction of such catheters over guidewires which have been pre-positioned within the urethra or other body lumen. At least some of these objectives will be met by the inventions described below.

2. Description of the Background Art

The use of an everting sleeve composed of thin, tensilized polytetrafluoroethylene for introducing catheters to body lumens is described in U.S. Pat. Nos. 5,531,717; 5,676,688; 5,711,841; 5,897,535; 6,007,488; 6,240,968; and EP605427B1. Other catheters employing everting sleeves for a variety of purposes are described in commonly assigned, copending application Ser. Nos. 10/794,337 (Attorney Docket No. 021807-000300US), filed on Mar. 5, 2004, 10/794,317 (Attorney Docket No. 021807-000400US), filed on Mar. 5, 2004, and 10/886,886 (Attorney Docket No. 021807-000800US), filed on Jul. 7, 2004, the full disclosures of which are incorporated herein by reference.

### BRIEF SUMMARY OF THE INVENTION

The present invention provides systems and methods for dilating and/or accessing body lumens within a patient's body. The body lumens will typically be natural and have a natural access orifice, but in other cases could be fully enclosed body lumens or systems or even body lumens which are created by penetrations or other artificial means. A preferred body lumen which may be treated or accessed by the present invention is the urethra, particularly the male urethra which is the exemplary embodiment below. Other natural body lumens which may be accessed and treated by the present invention include the ureter, hepatic ducts, cystic ducts, the cervical canal, fallopian tubes, the pulmonary bronchi, nasal passages, and the like. Exemplary closed luminal systems include the patient's vasculature, including both the arterial and venous vasculature, the meninges which circulates the spinal fluid, lymph circulation, and the like. Created body lumens include tissue tracts which are formed by needle penetrations, arterial-venous fistulas, and the like. In some cases, the body lumen being treated may be reached at least partially through another body lumen, such as treating a ureter which has been accessed through the urethra. In other cases, the body lumen can be accessed using laparoscopic, thoracoscopic, or other endoscopic techniques.

An exemplary technique and system for access and/or dilation of the urethra will be described hereinafter. It will be appreciated, however, that the principles and embodiments of the present invention may be applied to a much wider variety of target locations and access routes.

In a first aspect of the present invention, a luminal dilator assembly comprises an access sheath, an obturator, and an evertable lubricous sleeve. The access sheath has a proximal end and a distal end with a central passage for removably receiving the obturator. The obturator also has a proximal end, a distal end, and an axial passage. The evertable lubricous sleeve is received within the axial passage of the obturator and is positioned so that it everts over the distal ends of both the obturator and the sheath as the dilator is introduced through an orifice into a body lumen. Typically, the access sheath includes a proximal hub having an axial port through which the obturator can pass. Usually, the proximal hub includes a side port for fluid introduction into the central passage of the access sheath. In other instances, the side port can deliver fluid into the axial passage of the obturator, typically by having an aligned hole within the obturator wall.

The dimensions and materials of the luminal dilator will be selected pending on the intended use. Typically, the sheath will have a length in the range from 5 cm to 90 cm, a maximum outer width in the range from 3 mm to 12 mm, and a wall thickness in the range from 0.5 mm to 2 mm. The distal region of the sheath wall will typically be thinned or tapered to provide a smooth transition between the distal end of the dilator which extends beyond the distal end of the sheath (when the dilator is fully nested within the sheath), typically by a distance in the range from 0.2 cm to 2 cm. The access sheath will typically be extruded or otherwise formed from a suitable polymer, such as a polyethylene, a polypropylene, a polyvinyl chloride, a polyurethane, a polyester, a polyether block amide, or the like. The sheath alternatively could be a flexible composite of a metal coil or braid reinforced flexible composite with a highly elastic polymer such as silicone or polyurethane, or made rigid using polymers such as polycarbonate, polysulfone, nylon, or metal tubing such as stainless steel. The sheath could also be constructed of any combination of the above.

The obturator will have dimensions and be configured to be insertable and removable through a proximal end of an access sheath, usually through the axial port of the proximal hub. The obturator will usually include a handle having a threaded or otherwise modified end which can mate and attach to the proximal hub in order to firmly attach the obturator to the access sheath so that they can be advanced as a single dilator unit. For introduction to the urethra, the distal end of the obturator will usually be bendable, deflectable, and/or have a bend or deflection preformed over the distal most 1 cm to 2 cm, and the distal end of the obturator may be tapered, blunt, rounded, or the like to both facilitate introduction through the body lumen and to be compatible with eversion of the evertable lubricous sleeve, as described in more detail below.

The dimensions and materials of the obturator will be chosen to be compatible with the access sheath, the evertable lubricous sleeve, and to provide the mechanical characteristics suitable for introduction through and dilation of the target body lumen. Typically, the obturator will be composed of a flexible polymer chosen from the group consisting of polyethylene, polypropylene, polyvinyl chloride, polyolefin copolymers, acrylonitrile butadiene styrene, polytetrafluoroethylene, silicone, and the like. The polymer will typically have a hardness on the shore A range from 50 to 100, preferably from 70 to 95. Such soft materials may cause undesirable friction between the obturator tip and the lubricious sleeve as the lubricious sleeve is being everted, so it may be desirable to reduce the relative friction. The hardness may be adjusted in a variety of ways, e.g. by coating, coextruding or fusing different materials onto or over at least the tip of the obturator to reduce the relative friction. For example, the distal tip and outer surface of the obturator (e.g the eversion surface having the highest friction) may be hardened to a shore hardness in the range from 90 A to 100 A. The obturator may be coil or braid reinforced. Alternately, the obturator could be constructed of a rigid polymer or metal and slit, hinged, formed into a coil or otherwise articulated to allow deflection.

The axial passage will usually be aligned centrally with an obturator and will thus be coaxial with the central passage of the axis sheath when the obturator is present within the sheath. Alternatively, the axial passage of the obturator may be laterally offset and, in some instances, may be at least partially open along all or a portion of its axial length. In the latter case, the axial passage through the obturator will be partially enclosed by the inner surface of the access sheath when the obturator is present in the access sheath. In all cases, the axial passage of the obturator may be adapted to receive a guidewire. Preferably, the obturator has a length in the range from 6 cm to 100 cm, a maximum outer width in the range from 2.5 mm to 11.5 mm, and a central passage diameter in the range from 1 mm to 10 mm.

The evertable lubricous sleeve is adapted to initially be received or otherwise stowed within the axial passage of the obturator and to evert over the distal ends of the obturator and the sheath as the luminal dilator assembly is advanced through the target body lumen. The evertable lubricous sleeve typically comprises a polymeric tube having a length in the range from 5 cm to 90 cm, an inner diameter in the range from 2 mm to 12 mm, and a wall thickness in the range from 0.1 mm to 0.05 mm. The polymer is preferably a lubricious polymer and/or may be lubricated. Exemplary polymers include polytetrofluoroethylene, polyethylene, perfluoroalkoxy, polyurethane, perfluoromethylvinylether, perfluoropropylvinylether, and the like. A particular preferred polymer comprises tensilized polytetrofluoroethylene/perfluoropropylvinylether copolymer, such as that described in U.S. Pat. No. 6,240,968, the full disclosure of which is incorporated herein by reference. Optionally, the exterior of the access sheath and/or the surface of the evertable lubricious sleeve may be treated to reduce relative friction. For example, the exterior of the access sheath may be lubricated, hardened, and/or texturized.

In preferred embodiments, the evertable lubricous sleeve will include an anchor structure which is maintained outside of the axial passage of the obturator prior to deployment of the sleeve. The anchor will preferably include an anchor having an opening which allows the sheath-obturator combination to be advanced therethrough to evert the sleeve. An anchor is usually held in one hand by the physician and/or immobilized against the patient as the sheath-obturator combination is pushed through the opening. The anchor in turn immobilizes one end of the sheath, holding the deployed sheath stationary relative to the body lumen as the sheath-obturator is advanced. The sleeve thus provides a sterile barrier, inhibits axial displacement of the lumen tissue (by converting the axial motion of the dilator tip to a lateral opening force) and generally facilitates problematic entries into body lumens having strictures, occlusions, or other obstructions. After the sheath-obturator is fully advanced relative to the anchor, the obturator will usually be removed leaving the central passage of the sheath available for drainage, access, and other diagnostic and therapeutic procedures.

The systems and assemblies described above can be used in a variety of procedures for accessing a body lumen, dilating a body lumen, or combinations thereof. In a first aspect of the methods of the present invention, a body lumen may be accessed (but not necessarily dilated) by first positioning a sheath-obturator device at an opening to the body lumen. The sheath-obturator device is then advanced through the body lumen (typically through an anchor as described above) to evert a lubricious sleeve (attached to the anchor at one end) from an axial passage of the obturator over the exterior of the sheath-obturator. By then removing the obturator from the sheath, an axial passage through the sheath will be left in place to provide the desired access to a target site within or beyond the body lumen. A free end of the lubricious sleeve is preferably immobilized using the anchor so that the sleeve remains stationary relative to an inner wall of the body lumen, as generally described above. Optionally, the sheath-obturator device may be advanced over a pre-positioned guidewire to facilitate entry and passage past strictures, occlusions, and any other obstructions which may be present in the body lumen. The body lumen may be a natural or created body lumen as described above, preferably being a urethra, a ureter, a blood vessel, a hepatic duct, a cystic duct, a cervical canal, a fallopian tube, or the like.

The present invention further provides methods for dilating a body lumen even when access is not desired. Such methods may utilize the obturator of the systems as described above, but not necessarily in combination with a separate access sheath. The obturator is positioned at an opening to the body lumen, and the obturator is advanced through the body lumen to evert a lubricious sleeve from an axial passage of the obturator over an exterior of the obturator. The width and/or cross-sectional area of the obturator will be greater than that of the body lumen, narrowing, orifice, sphincter or stricture prior to dilation. Dilation may be effected by advancing the obturator into and expanding the tissue. In some cases, such as scarred strictures, the lumen tissue will be stretched beyond its elastic limits and achieve immediate permanent dilation. In other cases, the obturator, or sheath may be left in place within the body lumen for a time sufficient to achieve long term dilation, typically in the range from a few seconds to 30 min, often in the range from a couple of seconds to approximately one minute. Usually, but not necessarily, the lubricious sleeve will be left in place over the obturator while the obturator remains in the body lumen. The lubricious sleeve is advantageous as it continues to provide a sterile barrier during the dilation and remains in place to facilitate removal of the dilator when the dilation is complete. Still further optionally, a liquid, gas, or other fluid may be introduced between the sleeve and obturator during the dilation to help effect dilation or provide other desired therapeutic treatments. The fluid may be heated, or in some cases may be medicated. In the latter cases, it may be desirable to provide pores, microholes, or other means for releasing the medication through the sheath along all or a portion of the length of the sleeve. Thus, the methods and apparatus of the present invention are suitable for providing controlled drug and medication delivery to body lumens in a highly atraumatic manner.

In a still further aspect of the methods of the present invention, a sheath, much in the same form as the obturator in the previous example may be advanced into a body lumen over a pre-positioned guidewire. The guidewire may be pre-positioned through a suitable body orifice into the body lumen, and the sheath may then be advanced over the guidewire through the body lumen during the course of which a lubricious sleeve is everted from a central passage of the sheath and over the exterior of the dilator. The axial lumen of the sheath is sized so that the guidewire may fit through the center of the lumen, surrounded by a freely mobile sleeve. Thus, the guidewire and the sleeve may move independently of one another. It will be apparent to one skilled in the art that this is required as the guidewire and the everting sleeve must move in opposite directions as the device travels deeper into the lumen. When a tapered dilator is also employed, the lubricious sleeve may be stowed in the axial passage of the dilator, generally as described above. All other aspects of advancing the sheath will be analogous to those described above for advancing the dilators and the sheath-dilators of the present invention.

### BRIEF DESCRIPTION OF THE DRAWINGS

FIG. 1 illustrates a Memcath™ luminal access catheter of the prior art.

FIG. 2 is a perspective view of a luminal access and dilation system constructed in accordance with the principles of the present invention shown with the components disassembled.

FIG. 3 is a detailed view of a distal end of the luminal access and dilation system of FIG. 2, shown with the components assembled.

FIGS. 4A-4C are alternative cross-sectional views taken along line 4-4 of FIG. 3.

FIGS. 5A-5C are alternative views of a distal end configuration of an obturator of the system of FIG. 2.

FIGS. 6A and 6B illustrate alternative configurations for a proximal hub and handle assembly of the system of FIG. 2.

FIGS. 7A-7F illustrate use of the luminal access and dilation system of the present invention for dilating and accessing a urethra.

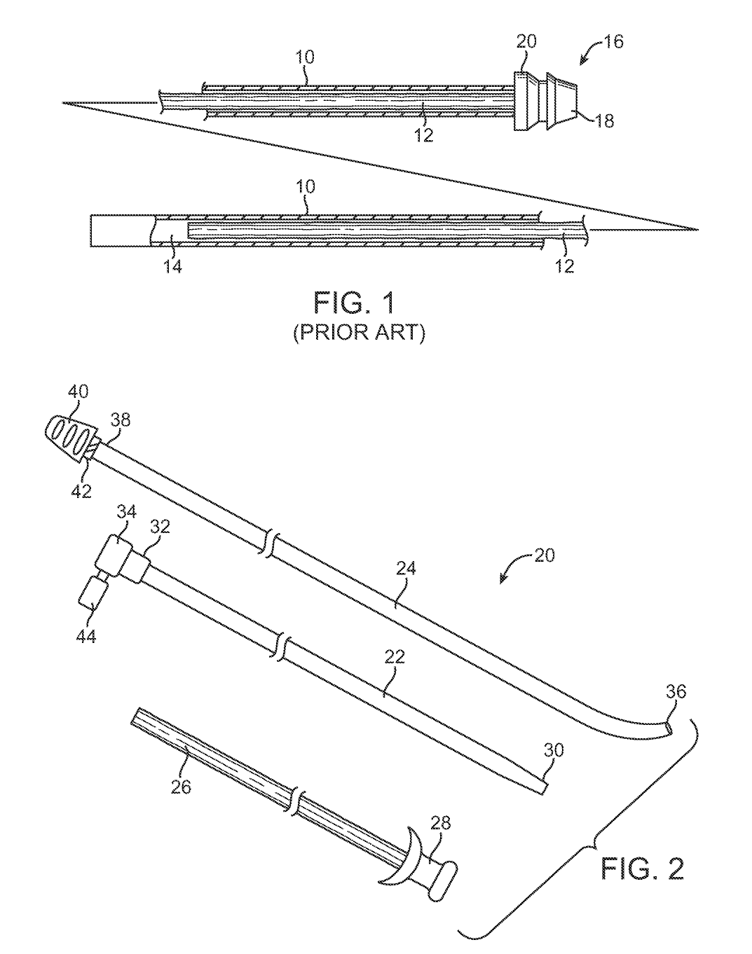

### DETAILED DESCRIPTION OF THE INVENTION

The systems and methods of the present invention are useful for providing access to and/or dilation of any natural or created body lumen of a patient where it is desired to temporarily or permanently place an access tube or a structure, perform a diagnostic or therapeutic procedure, or simply dilate the lumen. Most commonly, the systems and methods will be used to place a drainage, infusion, or other interventional tube or instrument through a natural body lumen to a target site within the body lumen or a hollow body organ connected to the natural body lumen. The methods described in the following description are directed specifically at dilating a urethra and/or accessing a bladder through the urethra It will be appreciated, however, that the principles of the present invention will apply more broadly as discussed above.

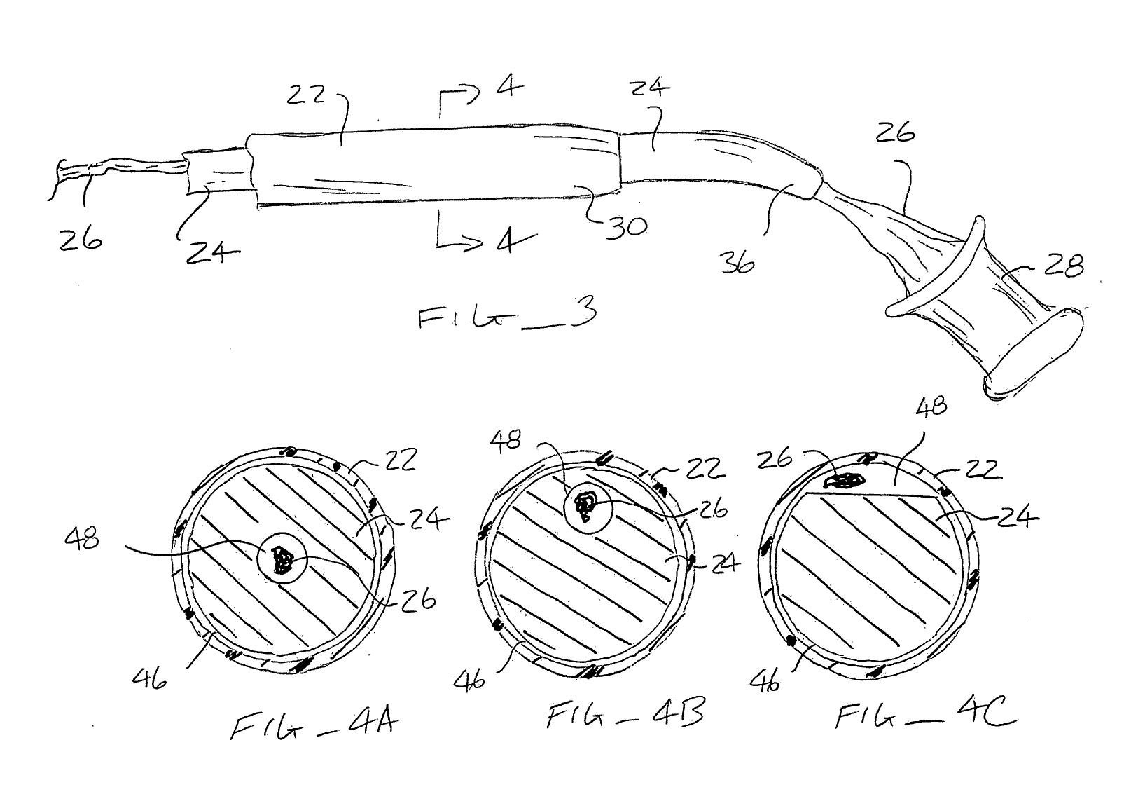

Referring now to FIGS. 2 and 3, an exemplary luminal access and dilation system 20 constructed in accordance with the principles of the present invention comprises an access sheath 22, an obturator 24, and an evertable lubricous sleeve 26. The evertable lubricous sleeve 26 has an anchor 28 at one end thereof while the other end is usually free of attachments. The access sheath 22 has a distal end 30 and a proximal end 32 with an attached proximal hub 34. The obturator 24 also has a distal end 36 and a proximal end 38 having an attached handle 40. The handle 40 has a threaded or other coupler 42 which allows the handle to be removably secured to the proximal hub 34 of the access sheath so that the obturator and access sheath can be attached and manipulated as a single assembly or unit. The proximal hub 34 has a side port 44 to permit fluid infusion and/or aspiration from either the obturator or the access sheath, as will be described in greater detail below with respect to FIGS. 4A-4C. The handle 40 may also incorporate a through hole or fluid port in direct communication with the axial lumen of the obturator for the same purpose.

As best seen in FIG. 3, the obturator 24 is received within an axial passage 46 (FIGS. 4A-4C) of the access sheath 22. The distal end 36 of the obturator preferably protrudes from the distal end 30 of the access sheath 22, preferably by a distance as set forth hereinabove. The distal end 36 of the obturator may be bent or deflected, as shown, or in other instances could be straight. The evertable lubricous sleeve 26 is preferably stowed within an axial passage 48 of the obturator 24, as best seen in FIGS. 4A-4C. As shown in FIG. 4A, the axial passage 48 may be a coaxial lumen or passage formed down the center of the obturator 24. Alternatively, the axial passage 48 could be laterally offset, as shown in FIG. 4B, or could even be an open region formed between one side of the obturator 24 and the inner surface of the access tube 22, as shown in FIG. 4C. Typically, the evertable lubricous sleeve will typically be backloaded through the axial passage 48 through the distal end 36 of the obturator so that the anchor 28 is generally located near the distal end when the luminal access and dilation system 20 is ready for use.

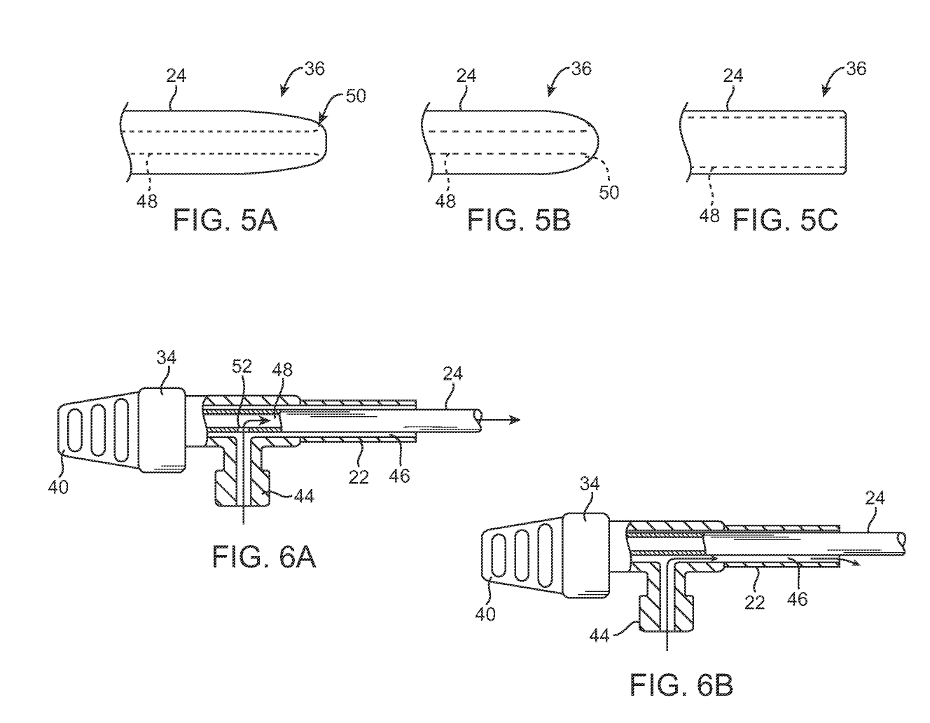

The distal end 36 of the obturator 24 may be configured in a variety of ways, some of which are shown in FIGS. 5A-5C. In a preferred embodiment, obturator 24 will have a slightly tapered and rounded distal end, so that a relatively small diameter aperture 50 is provided for outward eversion of the evertable lubricous sleeve. In other instances, the distal end 36 could have a more hemispherical distal end, again including aperture 50. The aperture and cross section of the obturator can be rounded, oval or polygonal and may be asymmetric. In a third, generally less preferred embodiment, the distal end 36 may be blunt, generally as shown in the prior art access sheaths described in U.S. Pat. No. 6,240,968, previously incorporated herein by reference.

Referring now to FIGS. 6A and 6B, the proximal end of the luminal access system 20 will initially have handle 40 of the obturator coupled to proximal hub 34 of the access sheath so that the obturator and access sheath cannot move axially relative to each other as the system is introduced to a body lumen, as described in more detail below. In this configuration, the obturator 24 is positioned in the central passage 46 of the access sheath 22, typically leaving a small annular space or clearance between the two system components. Fluid, either liquid or gas, can be infused through or aspirated from the system 20 in at least two ways, depending on the particular configuration of the system components. As illustrated in FIG. 6A, the dilator 24 may include an aperture 52 which may be aligned with the side port 44 to permit fluid to be infused through the axial passage 48 of the obturator. Alternatively, as shown in FIG. 6B, fluid infused through side port 44 may pass directly into the annular space in central passage 46 and/or channels on the surface of the obturator 24. Systems can be designed, of course, to permit infusion and aspiration simultaneously through both the central passage 46 of the access sheath and the axial passage 48 of the obturator.

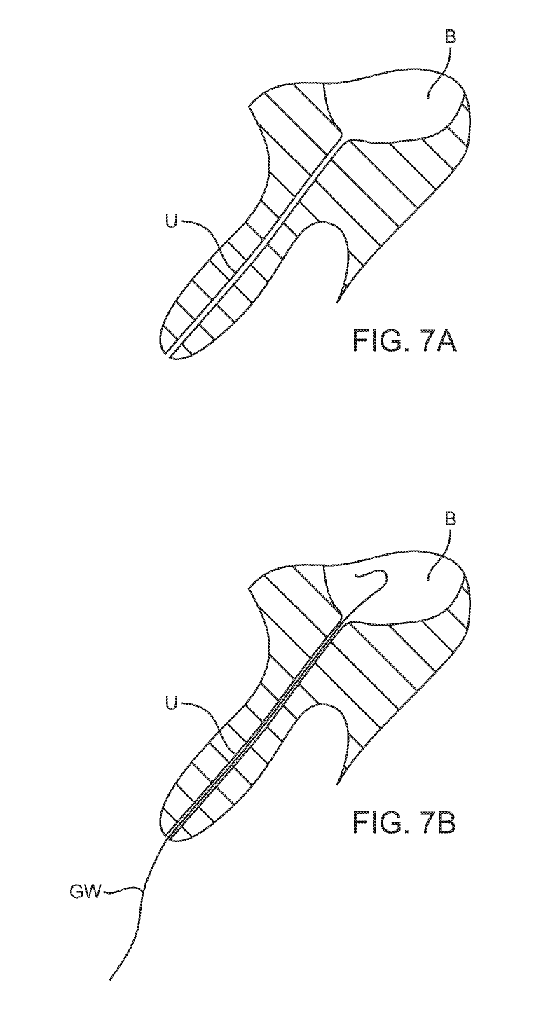

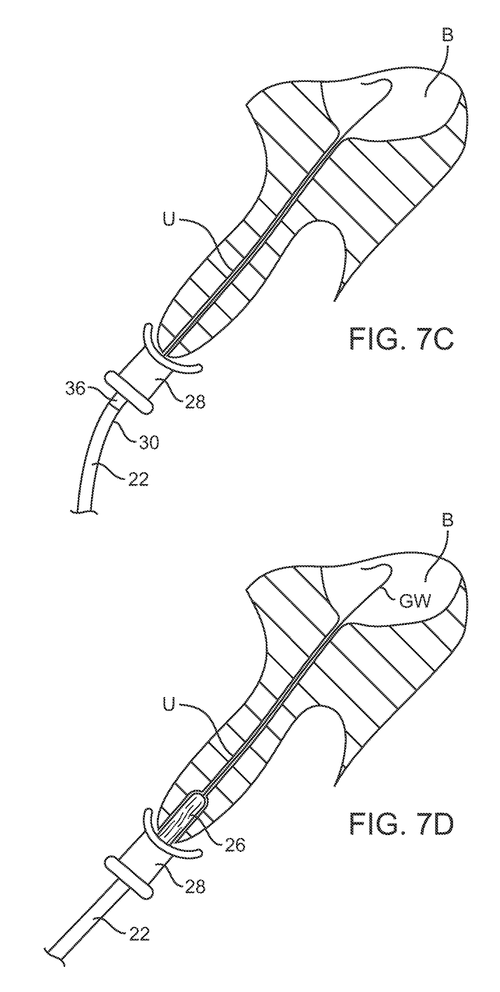

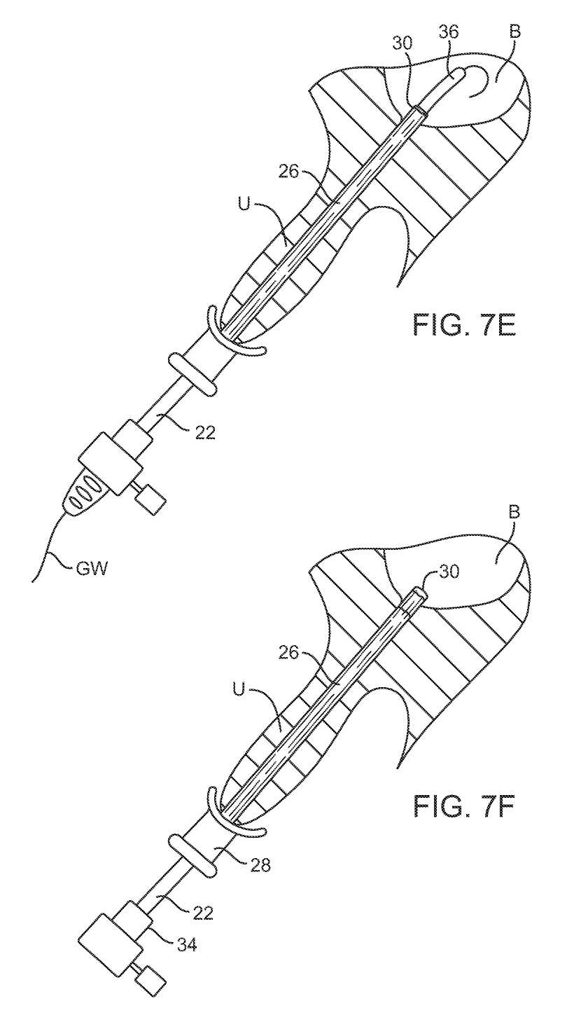

Referring now to FIGS. 7A-7F, use of the luminal access and dilator system 20 of the present invention for accessing a urethra in a male patient is illustrated. The urethra U in a generally collapsed and non-dilated configuration is shown in FIG. 7A. Optionally, but not necessarily, a guidewire GW can be introduced through the urethra and into the bladder B as shown in FIG. 7B. Either with or without the guidewire, the anchor 28 will initially be engaged against the entry to the urethra U, as shown in FIG. 7C. The assembly of the obturator 24 and access sleeve 22 is then distally or forwardly advanced through the urethra U toward the bladder B as shown in FIG. 7D. Such forward advancement causes the evertable lubricous sleeve 26 to be everted and drawn from the axial passage 48 (FIGS. 4A-4C) and the obturator 24 (FIG. 2), as shown in FIG. 7D. The obturator 24 and access sheath 22 may continue to be advanced until the distal end 36 of the obturator reaches the bladder B, as shown in FIG. 7E. Preferably, the distal end 30 of the access sheath will enter the bladder B, and the evertable lubricous sleeve 26 will cover the entire length of the access sheath which is in the urethra U.

If the purpose of the urethral access is merely to dilate the urethra, then the entire assembly of the dilator 24 and access sheath 22 may be withdrawn after the treatment is completed. In such cases, no separate access sheath is required, and obturator 24 could be used without such a sheath.

If the principal purpose of the procedure, however, is to provide access, then the obturator 24 may be withdrawn in a proximal direction from the access sheath 22, leaving the central passage 46 (FIGS. 4A-4C) of the access sheath available for access through the urethra U, as seen in FIG. 7F. Such access may be utilized for drainage, cystoscopy, introduction of other therapeutic or diagnostic tools, including tools for accessing the ureter and subsequent introduction into the kidney, or the like.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A luminal dilator assembly comprising:
an access sheath having a proximal end, a distal end, and a central passage therethrough;
an obturator having a proximal end, a distal end, and an axial passage therethrough, wherein said obturator is removably insertable into the central passage of the access sheath; and
an evertable lubricious sleeve which is received in the axial passage of the obturator and which everts over the distal ends of the obturator and the sheath.

2. A luminal dilator assembly as in claim 1, wherein the access sheath includes a proximal hub having an axial port through which the obturator can pass.

3. A luminal dilator assembly as in claim 2, wherein the proximal hub has a side port for fluid introduction into the central passage of the access sheath.

4. A luminal dilator assembly as in claim 2, wherein the proximal hub has a side port aligned with a hole in a side of the obturator to deliver fluid to the axial passage.

5. A luminal dilator assembly as in claim 1, wherein the access sheath has a length in the range from 5 cm to 90 cm, a maximum outer width in the range from 3 mm to 12 mm, and a wall thickness in the range from 0.5 mm to 2 mm.

6. A luminal dilator assembly as in claim 1, wherein a wall of the distal end of the access sheath is thinned or tapered.

7. A luminal dilator assembly as in claim 1, wherein the access sheath is composed of a polymer selected from the group consisting of a polyethylene, a polypropylene, a polyvinyl chloride, a polyurethane, a polyester, polyether block amide, a polycarbonate, a polysulfone, a polyetheretherketone, a silicone and any of these reinforced with coil or braid.

8. A luminal dilator assembly as in claim 2, wherein the obturator includes a handle adapted to releasably mate with the hub on the access sheath.

9. A luminal dilator assembly as in claim 1, wherein the axial passage of the obturator is aligned centrally in the obturator.

10. A luminal dilator assembly as in claim 1, wherein the axial passage of the obturator is laterally offset in the obturator.

11. A luminal dilator assembly as in claim 10, wherein the axial passage is at least partly open to the central passage of the access sheath.

12. A luminal dilator assembly as in claim 1, wherein the axial passage of the obturator is adapted to receive a guidewire.

13. A luminal dilator assembly as in claim 1, wherein the distal end of the obturator is rounded.

14. A luminal dilator assembly as in claim 1, wherein the distal end of the obturator is tapered.

15. A luminal dilator assembly as in claim 1, wherein the distal end of the obturator is blunt.

16. A luminal dilator assembly as in claim 1, when the obturator has a length in the range from 6 cm to 100 cm, a maximum outer width in the range from 2.5 mm to 11.5 mm, and diameter of the central passage in the range from 1 mm to 10 mm.

17. A luminal dilator assembly as in claim 1, wherein the obturator is composed of a polymer selected from the group consisting of polyethylene, polypropylene, polyvinyl chloride, polyolefin copolymers, acrylonitrile butadiene styrene, and polytetrafluoroethylene.

18. A luminal access system as in claim 1, wherein the sleeve comprises a polymeric tube.

19. A luminal access system as in claim 18, wherein the polymeric tube has a length in the range from 5 cm to 90 cm, an inner diameter in the range from 2 mm to 12 mm, and a wall thickness in the range from 0.01 mm to 0.05 mm.

20. A luminal access system as in claim 18, wherein the polymer comprises a lubricious polymer.

21. A luminal access system as in claim 18, wherein the polymer is lubricated.

22. A luminal access system as in claim 13, wherein the polymer is selected from the group consisting of polytetrafluoroethylene (PTFE), polyethylene (PE), perfluoroalkoxy (PFA), polyurethane (PU), perfluoromethylvinylether (PMFA), and perfluoropropylvinylether (PPVE).

23. A luminal access system as in claim 22, wherein the polymer comprises tensilized PTFE/PPVE copolymer.

24. A luminal dilator assembly as in claim 22, wherein the polymeric tube is heat sealed to form a tube from a flat sheet.

25. A luminal dilator assembly as in claim 1, wherein a distal tip of the obturator extends distally of the distal end of the access sheath by a distance in the range from 0.5 cm to 5 cm when the proximal ends of the obturator and the access sheath are mated.

26. A luminal dilator assembly as in claim 1, further comprising a guidewire removably receivable in the axial passage of the obturator so that the assembly may be introduced over the guidewire.

27. A luminal dilator assembly as in claim 1, wherein the distal tip of the obturator has a reduced surface friction relative to the rest of the obturator to facilitate eversion of the sleeve.

28. A luminal dilator assembly as in claim 27, wherein the distal tip of the obturator is hardened to have a shore hardness in the range from 60 A to 100 A.

29. A luminal dilator assembly as in claim 27, wherein the exterior of the access sheath and/or the lubricious sleeve is treated to have a reduced friction to facilitate eversion of the sleeve.

30. A luminal dilator assembly as in claim 29, wherein the exterior of the access sheath is lubricated, hardened, and/or textured.

31. A method for dilating a body lumen, said method comprising:
positioning a sheath-obturator device at an opening to the body lumen;
advancing the sheath-obturator device through the body lumen to evert a lubricious sleeve from an axial passage of the obturator over the exterior of the sheath-obturator; and
removing the obturator from the sheath to leave an axial passage within the sheath.

32. A method as in claim 31, wherein advancing the sheath-obturator comprises immobilizing a free end of the lubricious sleeve so that the sleeve remains stationary relative to an inner wall of the body lumen as the sheath-obturator is advanced.

33. A method as in claim 32, wherein immobilizing a free end of the sleeve comprises holding an anchor attached to the free end adjacent to the opening to the body lumen as the sheath-obturator is advanced through the entry.

34. A method as in claim 31, further comprising positioning a guidewire through the entry and into the body lumen, wherein the sheath-obturator is advanced over a guidewire.

35. A method as in claim 31, wherein the natural body lumen is selected from the group consisting of a urethra, a ureter, a blood vessel, a hepatic duct, a cystic duct, a cervical canal, and a fallopian tube.

36. A method for dilating a body lumen in a patient, said method comprising:
positioning an obturator at an opening to the body lumen;
advancing the obturator through the body lumen to evert a lubricious sleeve from an axial passage of the obturator over the exterior of the obturator; and
leaving the obturator in place within the body lumen for a time sufficient to effect dilation of the body lumen.

37. A method as in claim 36, wherein the lubricious sleeve is left in place over the obturator while the obturator is left in place in the body lumen.

38. A method as in claim 37, further comprising circulating a fluid between the sleeve and the obturator.

39. A method as in claim 36, wherein advancing the obturator comprises immobilizing a free end of the lubricious sleeve so that the sleeve remains stationary relative to an inner wall of the body lumen as the obturator is advanced.

40. A method as in claim 39, wherein immobilizing a free end of the sleeve comprises holding an anchor attached to the free end adjacent to the opening to the body lumen as the obturator is advanced through the entry.

41. A method as in claim 36, further comprising positioning a guidewire through the entry and into the body lumen, wherein the obturator is advanced over a guidewire.

42. A method as in claim 36, wherein the natural body lumen is selected from the group consisting of a urethra, a ureter, a blood vessel, a hepatic duct, a cystic duct, a cervical canal, and a fallopian tube.

43. A method for advancing a sheath into a body lumen, said method comprising:
positioning the sheath at an opening to the body lumen;
advancing the sheath over a pre-positioned guidewire through the body lumen to evert a lubricious sleeve from a central passage of the sheath over the exterior of the sheath.

44. A method as in claim 43, wherein advancing the sheath comprises immobilizing a free end of the lubricious sleeve so that the sleeve remains stationary relative to an inner wall of the body lumen as the obturator is advanced.

45. A method as in claim 44, wherein immobilizing a free end of the sleeve comprises holding an anchor attached to the free end adjacent to the opening to the body lumen as the sheath is advanced through the entry.

46. A method as in claim 23, wherein the natural body lumen is selected from the group consisting of a urethra, a ureter, a blood vessel, a hepatic duct, a cystic duct, a cervical canal, and a fallopian tube.
